(19) United States
(12) Patent Application Publication
(10) Pub. No.: US 2006/0173461 A1
(43) Pub. Date: Aug. 3, 2006

(54) Cannulated orthopedic screw

(76) Inventors: David B. Kay, Akron, OH (US); Lee A. Strnad, Broadview Heights, OH (US); Dustin Ducharrme, Akron, OH (US)

Correspondence Address: HUDAK, SHUNK & FARINE, CO., L.P.A., 2020 FRONT STREET, SUITE 307, CUYAHOGA FALLS, OH, 44221, US

(21) Appl. No.: 11/340,026

(22) Filed: Jan. 26, 2006

Related U.S. Application Data
(60) Provisional application No. 60/648,209, filed on Jan. 28, 2005.

(51) Int. Cl. A61B 17/58

(52) U.S. Cl. 606/73

(57) ABSTRACT

The present invention relates to a cannulated orthopedic screw having a torque driving head with a spherical wall for multiaxial use, a rounded fluted insertion tip and a threaded portion including a cancellous thread contiguous with a non-threaded portion. The threaded portion has a major diameter defined by a spiraling cancellous type thread and a minor diameter. The screw includes a multilobe torque driving recess.

THIS APPLICATION IS BASED ON U.S. PROVISIONAL APPLICATION SERIAL No. 60/648,209, FILED ON JAN 28, 2005

### FIELD OF THE INVENTION

The present invention relates to an orthopedic screw for use repair or reconstruction of bones.

### BACKGROUND OF THE INVENTION

Screws are commonly used for fixation in orthopedic procedures; to fix a plate, rod, or other construct to a bone or alone to allow two joined bone fragments to fuse. Wires can also be used to draw fragments together, or to hold them together to allow fusion. Cannulated screws combine some of the advantages of both K wires and solid screws. They can be used to draw fragments together and to hold them in a sufficiently close relationship to enable fusion to occur. They also permit the advantage of pre-aligning the bone pieces, or fragments using a wire and subsequently loading the bones by tightening the screw in relation to the wire. The hollow channel reduces the weight, and enables other advantages.

The present invention provides a cannulated screw that is an improvement over those of the prior art and is useful for fixation by itself, or in conjunction with other constructs. The present invention could serve for the treatment of a broad range of indications including relatively straightforward fracture repair following trauma in an otherwise healthy individual where screws are used alone or with plates to maintain the integrity of the bones while they heal, as well as for more complex surgeries such as reconstruction to correct congenital or age related deformation. Reconstruction often includes arthrodesis or partial or total fusion which involves removal of a joint and the use of a mechanical-biological construct to keep the bones immobile while fusion occurs. Further orthopedic surgeons may be called upon to achieve soft-tissue balancing by readjusting the length of tendons and ligaments or to reshape the bone itself through removal or repositioning in a procedure known as an “osteotomy”. In an aging or diabetic population, these procedures may also involve dealing with the difficulties of poor quality bone and/or compromised soft tissue.

These surgeons typically include small bone specialists such as hand surgeons and feet and ankle and podiatric surgeons, but can also include general orthopedic surgeons who may be called upon to perform procedures which would benefit from the use of a cannulated screw, including, for example, the repair of femoral neck fractures, tibial fractures, humeral fractures. A particularly advantageous small bone application is for the repair of calcaneal fractures.

The present invention provides a cannulated screw for use alone or along with of a construct which could include a plate and screws. The screw is designed specifically for the small bone market, i.e. for use in bones distil to the elbow and knee, including, for example, the ulna, radius, tibia, fibula, as well as the metacarpals, carpals, metatarsals, and tarsals and phalanges. The screw can be used in applications previously mentioned, for example those that require fixation within a single bone such as the stabilization of a fracture or the screw can be used across two or more bones so as to facilitate total or partial fusion.

The screws are self-tapping screws including a cannulation. The internal recess provided by the cannulation can be used as a place to press fit a screw holder in an instrument or can be used for additional fixation, for example using a wire. The screws include a blunt cutting end having multiple flutes, and preferably 2 or 3, and most preferably 3 flutes at the insertion tip and which extend up the shaft toward the head for a distance of between about 1 and 4, and preferably between 1.5 and 3 rotations of the thread. The screws further include a cancellous type thread which has been modified for bite. The screws have a distal threaded portion which extends between about a quarter and three quarters, and preferably about a third to about a half of the way up the shaft from insertion tip toward the head, and an unthreaded proximal portion. The screws are of particular advantage in that they provide for an excellent bite in the distal bone and can be used to compress that bone toward a proximal bone segment which engages the unthreaded portion of the screw.

The head is a rounded head having a multilobed torque driving recess. The screws further include a torque driving recess that may be a hexagon, a sinusoidal shape, or a modification of a sinusoidal (multilobed) shape which preferably has 4-8, and preferably 6 sinusoidal lobes. The recess can be of a constant size in the direction of the longitudinal axis, or can taper inward along the longitudinal axis of the screw toward the bottom of the recess. In addition, the head of the screw can include a rounded portion or spherical shaped head to permit multiaxial insertion, i.e. in a corresponding rounded or spherical recess in a countersunk screw hole in a plate or other construct. The screws can be provided in typical lengths for small bone use, i.e. from about 10 mm to about 150 mm and typically in standards lengths in 5 or 10 mm increments from 40 mm to 100 mm with a major diameter of about 2.0 to 8.0 mm. The screws include a constant thread pitch. The screws can be made of appropriate biocompatible material, including for example surgical grade stainless steel and titanium.

### BRIEF DESCRIPTION OF THE DRAWINGS

FIG. 1 is a side view of a solid orthopedic screw in accordance with a first embodiment of the invention;

FIG. 2 is a cross-section of the screw taken along line 2-2;

FIG. 3 is a top perspective view of the screw;

FIG. 4 is a bottom perspective view of the screw;

FIG. 5 is a top view of a plate which could be used with the screw of the present invention;

FIG. 6 is a cross section taken along line 6-6 of the plate shown in FIG. 5.

FIG. 7 is a side view of an orthopedic screw in accordance with a second embodiment of the invention;

FIG. 8 is a cross-section of the screw taken along line 8-8;

FIG. 9 is a bottom view of the screw of FIG. 8;

FIG. 10 is a cross-section of the screw taken along line 10-10;

FIG. 11 is a top view of the screw of FIG. 8;

FIG. 12 is a detail of the thread of the screw of FIG. 8; and

FIG. 13 is a top perspective view of the screw of FIG. 8.

### DETAILED DESCRIPTION OF THE INVENTION

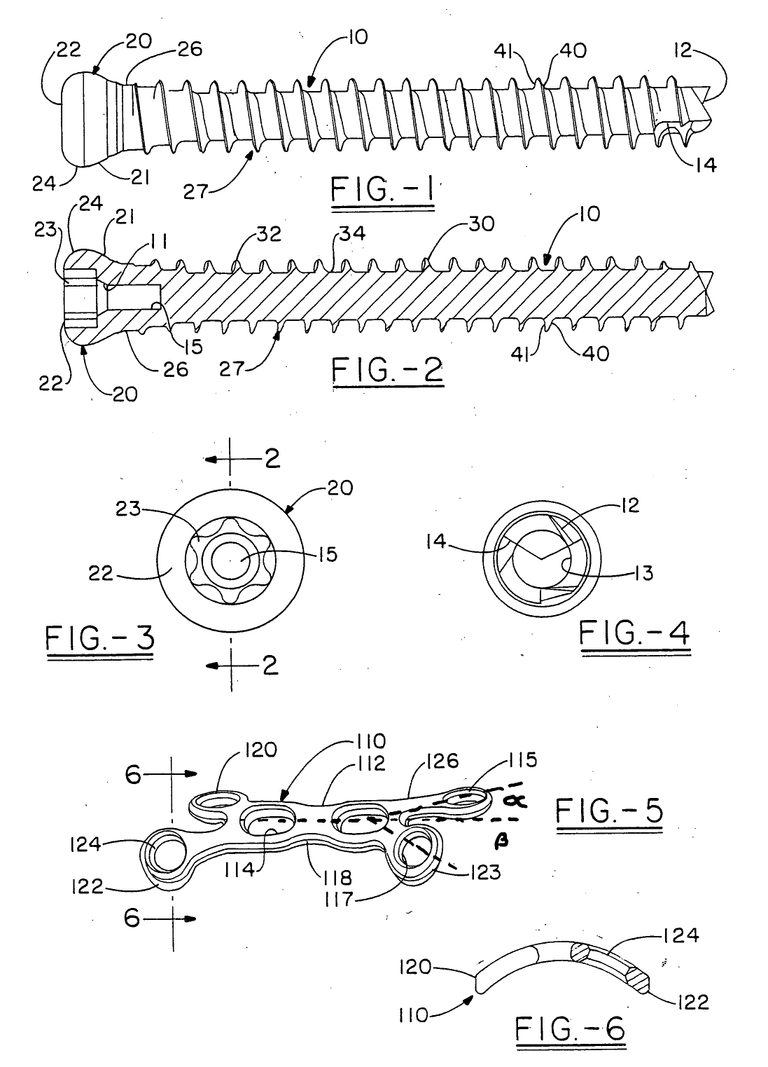

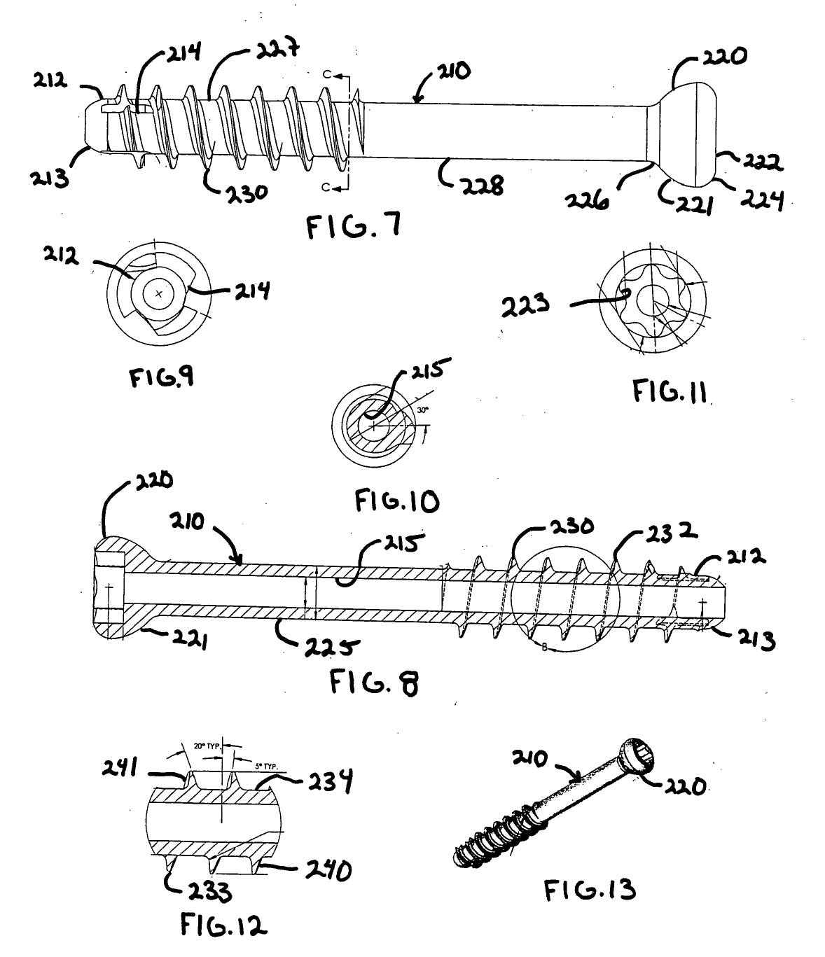

FIGS. 1-4 show a first embodiment of an orthopedic screw 10 in accordance with the present invention. The distal end of the screw includes a cutting tip 12 which is self-starting and self-tapping. The term “distal” is used herein to mean the end that would be farthest from the point of attachment to a plate if one were used, i.e. the insertion tip, and “proximal” is used to mean the opposite end of the screw, i.e. the head. The cutting tip 12 is provided by a conical recess 13 and a plurality of flutes 14 or grooves that form sharp cutting surfaces at the terminus of the screw. The screw 10 can include a partial or full cannula 15 along its longitudinal axis. While the screw is shown as including a cannula in the form of a through bore in the drawings, the bore can project only partially toward the distal end of the screw, or can be absent. In a preferred embodiment the screw includes the partial cannulation which is a cylindrical recess extending at least about 1.5 mm up to about 5 mm, and preferably about 2 mm to 4 mm based upon the diameter of the screw. An angled area 11 connects the cannulation or recess with the torque driving recess. The cannulation is used with a torque driving instrument that has a corresponding shaped post that will fit in the screw so that the screw is self-centering, is held in position on the torque driving instrument in a friction fit, and seats the screw so as to avoid stripping the interface between the screw and the torque driver.

The head 20 of the screw includes a rounded area 21 which preferably includes from about 0.75 mm to about 2.0 mm of a sphere having a diameter of from about 4 mm to about 5 mm. This defines a side wall which will allow for multi-axial placement in a screw hole, for example, in a plate that has a corresponding concavity. In the event that the screw is used alone, the rounded area eliminates sharp transitions between the threaded area and the head of the screw.

The screw head 10 has a relatively flat proximal surface 22 having radiused transitions 24 into the rounded area of the side wall of the head. The proximal surface includes a torque driving recess 23, such as a modified multilobe shape as is shown in FIG. 3. A necked area 26 joins the rounded area 21 of the head side wall to a threaded portion 27 of the screw. The threaded portion 27 includes a cancellous thread 30 with a constant major diameter 32 which is defined by the spiraling outer edge of the thread 33 which runs out to a very fine edge and a minor diameter 34 defined by the inner portion of the screw at the base of the thread. The minor diameter 34 is constant over a distil portion of the thread so as to define a cylinder with a spiraling thread. The minor diameter also includes a proximal portion that tapers inward over the length of the first four threads toward the distil end in order to improve fatigue strength and to improve compression at the proximal cortical bone interface and to compensate for bone re-adsorption. The tapered portion of the screw 36 includes a taper of from about 2° to about 20°, or more preferably from about 4° to about 12°, and most preferably about 60 to about 10° (i.e. about 8°) which tapers over from about 2 to about 10, and more preferably about 3 to about 6 complete turns (360°) of the thread 33. The pitch is between about 0.5 and 2.0 millimeters in length (i.e. a thread revolution of 360° per 0.5 to 2.0 millimeters).

The thread is a cancellous thread with a front thrust 40 surface having an angle of from about 10° to about 30°, or more preferably from about 15° to about 25°, and most preferably about 18° to about 22° (i.e. about 20°) to a plane perpendicular to the longitudinal axis of the screw, while the rear surface 41 forms an angle of about 0° to about 10°, or more preferably from about 0° to about 8°, and most preferably about 3° to about 7°(i.e., about 5°) to the plane perpendicular to the longitudinal axis of the screw.

The screw can be made from an appropriate biocompatible material having appropriate strength characteristics including surgical grade stainless steel or titanium or absorptive materials.

A plate with which the screw of the present invention can be used to advantage is shown in FIGS. 5 and 6. The plate 110 is shown having a modified x-shape or asymmetrical dog-bone shape with a central trunk portion 112 defining the longitudinal axis of the plate. The trunk portion 112 includes one or preferably more elongated screw holes 114 along the longitudinal axis. The number of screw holes will depend on the length of the plate, and may range from 0 to 6. The screw holes 114 are preferably elongated to allow the plate to be set initially and subsequently to be slide into a different position and tightened down. Further, the screw holes include annular rings 115 of increased thickness in the vertical direction about through bores 117. The through bores 117 in the trunk portion have a longitudinal axis that is perpendicular to plane tangent to the top radius of the plate. The area linking the screw holes has a decreased width so as to define a waist area 118 that will bend laterally (or “curve”) relative to the longitudinal axis and which will bend longitudinally to form a curved area in and out of the plane of the plate. This thinner area also facilitates twisting of the plate so as to allow the plate to spiral, or wrap around it longitudinal axis. The increased annular area around the screw holes resists deformation when a bending device is used to apply a force to the plate through the screw holes.

The plate 110 also includes at least one set, and preferably two opposing sets of arms 120. As viewed in FIG. 5, these sets of arms can be viewed as a set of upper 122 and lower arms 123, although it is understood that the orientation of the plate can vary even after the plate has been fixed to the bone so that the terms upper and lower are only used to distinguish the pair on one side of the trunk portion 112 from the pair on the other side of the trunk portion 112. Each of the arms in a set includes screw holes 124 which are placed at a radially equal distance but which diverging asymmetrically from the longitudinal axis of the plate 110. More specifically, each set of arms includes one arm that defines a smaller angle of divergence a from the longitudinal axis of the trunk portion than the angle of divergence of the other arm β. For example, the first angle shown in FIG. 1 at a may be from about 5° to about to 25°, and more preferably from about 10° to about to 20° and most preferably from about 12° to about to 16°, while the second angle shown at β from about 10° to about to 35°, and more preferably from about 15° to about to 30° and most preferably from about 22° to about to 26° with a preferred difference in the angles beings from about 2° to about to 20°, and more preferably from about 4° to about to 16° and most preferably from about 8° to about to 12°. On the inferior side, or the side that would be facing the bone surface in use, the arms continue the radius of curvature of the trunk portion. The superior or top side of the plate has a similar radius of curvature as the top surface of the plate has an outline that corresponds with the shape of the bottom of the plate (excluding the thickened annular area surrounding the screw holes.) The screw holes 124 are placed with the longitudinal axis perpendicular a tangent to the top surface of the arm with the effect that the longitudinal axes of the screws converge in the direction of the distil end. This increases the pull-out strength of the screws. Since the arms are asymmetrical relative to each other, and in particular since they diverge from the longitudinal axis of the trunk portion at differing angles, conflicts in the positions of paired screws is avoided so that the screws of a set of arms do not impinge on each other. This is even more important instances where the plate is bent around the longitudinal axis so as to wrap around the longitudinal axis of the bone.

The arms 120 also each include a screw hole 124 which, like the trunk portion 112 has a linking portion 126 that joins annular areas 125 of increased thickness that rings a through bore 127. Again this design facilitates the desired bending while resisting deformation of the screw holes 124 when they are used with the bending instrument to contour the plate. While the angle of the arms 120 of each one of a pair of a respective set of arms 122 and 123 varies so as to create a bilateral asymmetry, meaning that the plate is not symmetrical with respect to a plane that passes through the longitudinal axis in the vertical direction from the superior (the top side relative to the bone) to the inferior side (the side facing the bone), the “first plane”. However, the position of the arms in each set is preferably flipped so that the symmetry about a plane transverse to the first plane is a mirror image this is defined herein as transverse mirror symmetry. Further the length of each of the arms of a pair will vary so that the radial length of the center of the screw hole to the intersection with the longitudinal axis will be the same. As shown in FIG. 6, the plate includes a radial curve about the longitudinal axis. The radius is typically about 10 mm with a transverse dimension from the edge of one arm to the edge of the other arm of an upper or lower pair being about 15 or 16 mm, and the screw bore having a longitudinal axis of about 24° to a plane passing through the longitudinal axis of the plate. The bores are typically about 3.75 mm for a 3.5 mm diameter screw. In a further embodiment, the bore could be threaded.

FIGS. 8-13 show a second embodiment of an orthopedic screw 210 in accordance with the present invention. The distil end of the screw includes a cutting tip 212. The cutting tip 212 is provided at a blunt or rounded end 213 having a plurality of straight cutting flutes 214 or grooves that form sharp cutting surfaces at the terminus of the screw. The blunt end has a full spherical radius (meaning that except for the opening to the cannula, the end describes a sphere) that is intended to be minimally disruptive to the soft tissue at the distal end of the screw. The screw 210 includes a bore or cannula 215 along its longitudinal axis which is a cylindrical recess extending the length of the screw. An angled area 211 connects the cannulation or recess with the torque driving recess. The cannula is cylindrical and is from about 1 to 3 mm in diameter.

The head 220 of the screw includes a rounded area 221 which preferably includes from about 0.75 mm to about 5.0 mm of a sphere (i.e. in depth) having a diameter of from about 3 mm to about 9 mm. This defines a side wall which will allow for multi-axial placement in a screw hole, for example, in a plate that has a corresponding concavity. In the event that the screw is used alone, the rounded area eliminates sharp transitions between the threaded area and the head of the screw.

The screw head 220 has a relatively flat proximal surface 222 having radiused transitions 224 into the rounded area 221 of the side wall of the head. The proximal surface includes a torque driving recess 223, such as a modified multilobe shape as is shown in FIG. 11. A necked area 226 joins the rounded area 221 of the head side wall to the shaft 225 of the screw. The shaft 225 has a proximal area 228 that is cylindrical, and void of threads and a distal portion 227 which includes threads. The threaded portion 227 includes a cancellous thread 230 with a constant major diameter 232 which is defined by the spiraling outer edge of the thread 233 which runs out to a very fine edge and a minor diameter 234 defined by the inner portion of the screw at the base of the thread. The minor diameter 234 is constant over a distal portion of the thread so as to define a cylinder with a spiraling thread. The pitch is between about 0.5 and 3.0 millimeters in length (i.e. a thread revolution of 360° per 0.5 to 3.0 millimeters).

As for the first embodiment of the invention, the thread of the cannulated screw is a modified cancellous thread with a front thrust 240 surface having an angle of from about 10° to about 30°, or more preferably from about 15° to about 25°, and most preferably about 18° to about 22° (i.e. about 20°) to a plane perpendicular to the longitudinal axis of the screw, while the rear surface 241 forms an angle of about 0° to about 10°, or more preferably from about 0° to about 8°, and most preferably about 3° to about 7° (i.e., about 5°) to the plane perpendicular to the longitudinal axis of the screw. The screw also has a section adjacent the head that is free from threads in order to facilitate procedures in which the screw in inserted through a bone fragment into a second fragment and the second fragment is drawn into contact with the first fragment. The non-threaded portion extends from about a quarter to three quarters of the distance of the screw shaft, and preferably from about one half to about two thirds of this distance. These procedures are sometimes referred to as lag procedures.

While in accordance with the patent statutes, the best mode and preferred embodiment have been set forth, the scope of the invention is not limited thereto, but rather by the scope of the attached claims.

What is claimed is:

1. An orthopedic screw comprising:
a head and a shaft with a threaded portion, a non-threaded portion and an insertion tip and a longitudinal axis and having a cannulation along the longitudinal axis and having a major diameter defined by a spiraling thread and a minor diameter, the head having a surface including a torque driving recess and joined by a bevel to a rounded side wall and the major diameter of the screw remaining substantially the same along the length of the threaded portion, the insertion tip including a rounded tip and the tip having a plurality of flutes, the screw being made from surgical stainless steel or titanium.

2. An orthopedic screw as set forth in claim 1 wherein the head of the screw further includes a rounded side wall.

3. An orthopedic screw as set forth in claim 1 wherein the thread has a front thrust surface having an angle of from about 10° to about 30°, to a plane perpendicular to the longitudinal axis of the screw, and a rear surface which forms an angle of about 0° to about 10° to the plane perpendicular to the longitudinal axis of the screw.

4. An orthopedic screw as set forth in claim 3 wherein the front thrust surface forms an angle of from about 15° to about 25°, and the rear surface forms an angle of from about 0° to about 8°.

5. An orthopedic screw as set forth in claim 4 wherein the front thrust surface forms an angle of from about 18° to about 22°, and the rear surface forms an angle of from about 3° to about 7°.

6. An orthopedic screw as set forth in claim 5 wherein the front thrust surface forms an angle of from about 20°, and the rear surface forms an angle of from about 5°.

7. An orthopedic screw as set forth in claim 1 wherein the torque driving recess is a multilobe recess.
